(19) United States
(12) Patent Application Publication
(10) Pub. No.: US 2011/0098784 A1
(43) Pub. Date: Apr. 28, 2011

(54) Channel-Specific Loudness Mapping for Prosthetic Hearing Systems

(75) Inventors: Peter Schleich, Telfs (AT); Dirk Meister, Axams (AT)

(73) Assignee: MED-EL ELEKTROMEDIZINISCHE GERAETE GMBH, Innsbruck (AT)

(21) Appl. No.: 12/910,007

(22) Filed: Oct. 22, 2010

Related U.S. Application Data
(60) Provisional application No. 61/254,279, filed on Oct. 23, 2009.

(51) Int. Cl. A61F 11/04; A61N 1/36

(52) U.S. Cl. 607/57

(57) ABSTRACT

Generating electrode stimulation signals is described for stimulating cochlear tissue using an implanted electrode array. An acoustic audio signal is processed to generate band pass signals which represent an associated band of audio frequencies. Stimulation information is extracted from the band pass signals to generate stimulation event signals that define electrode stimulation signals. The stimulation event signals are then weighted according independent channel-specific loudness functions to produce electrode stimulation signals. The electrode stimulation signals are developed into output electrode pulses to the electrodes in the implanted electrode array for stimulating cochlear tissue.

This application claims priority from U.S. Provisional Patent Application 61/254,279, filed Oct. 23, 2009, which is incorporated herein by reference.

### FIELD OF THE INVENTION

The present invention relates to a mast for a wind turbine and a corresponding method for manufacturing such a mast.

### BACKGROUND ART

FIG. 1 shows major functional blocks in the signal processing arrangement typical of existing cochlear implant (CI) systems wherein some relatively large number N band pass signals containing stimulation timing and amplitude information are assigned to a smaller number M stimulation electrodes. Preprocessor Filter Bank 101 pre-processes an initial acoustic audio signal, e.g., automatic gain control, noise reduction, etc. Each band pass filter in the Preprocessor Filter Bank 101 is associated with a specific band of audio frequencies so that the acoustic audio signal is filtered into some N band pass signals, B₁ to B_{N} where each signal corresponds to the band of frequencies for one of the band pass filters.

The band pass signals B₁ to B_{N} are input to an Information Extractor 102 which extracts signal specific stimulation information—e.g., envelope information, phase information, timing of requested stimulation events, etc.—into a set of N stimulation event signals S₁ to S_{N}, which represent electrode specific requested stimulation events. For example, channel specific sampling sequences (CSSS) may be used as described in U.S. Pat. No. 6,594,525, which is incorporated herein by reference.

Pulse Weighting Module 103 applies a non-linear mapping function (typically logarithmic) to the amplitude of the each band-pass envelope. This mapping function typically is adapted to the needs of the individual CI user during fitting of the implant in order to achieve natural loudness growth. This may be in the specific form of weights that are applied to each requested stimulation event signal S₁ to S_{N} with a weighted matrix of stimulation amplitudes that reflect patient-specific perceptual characteristics to produce a set of electrode stimulation signals A₁ to A_{M} that provide and optimal electric tonotopic representation of the acoustic signal. Equation 1 shows a typical weighting matrix of size M×N:

$$W = ( \begin{matrix}1 & 0.923 & 0.846 & … & … & 0 & 0 & 0 \\ 0 & 0.077 & 0.154 & … & … & 0 & 0 & 0 \\ 0 & 0 & 0 & … & … & 0 & 0 & 0 \\ … & … & … & … & … & … & … & … \\ 0 & 0 & 0 & … & … & 0.154 & 0.077 & 0 \\ 0 & 0 & 0 & … & … & 0.846 & 0.923 & 1\end{matrix} ) \qquad Equation 1$$

Matrix weighting of the stimulation pulses is described further in U.S. patent application Ser. No. 12/427,933, filed Apr. 22, 2009, which is incorporated herein by reference. In such an arrangement, the stimulation event signals may be pooled into a smaller number of overlapping macro bands, and within each macro band the channel with the highest envelope may be selected for a given sampling interval, as described for example in U.S. Patent Application 61/145,805, filed Jan. 20, 2009, which is incorporated herein by reference.

Pulse Weighting Module 103 also controls loudness mapping functions. The amplitudes of the electrical pulses are derived from the envelopes of the assigned band-pass filter outputs. As shown in FIG. 2, a logarithmic function with a form-factor C typically may be applied to stimulation event signals S₁ to S_{N} as a loudness mapping function, which generally is identical across all the band pass analysis channels. In different systems, different specific loudness mapping functions other than a logarithmic function may be used, though still just one identical function is applied to all channels as shown in FIG. 3 to produce the electrode stimulation signals A₁ to A_{M} outputs from the Pulse Weighting Module 103.

Finally, patient specific stimulation is achieved by individual amplitude mapping and pulse shape definition in Pulse Shaper 104 which develops the set of electrode stimulation signals A₁ to A_{M} into a set of output electrode pulses E₁ to E_{M} to the electrodes in the implanted electrode array which stimulate the adjacent nerve tissue. Whenever one of the requested stimulation event signals S₁ to S_{N} requests a stimulation event, the respective number of electrodes is activated with a set of output electrode pulses E₁ to E_{M}.

### SUMMARY OF THE INVENTION

Embodiments of the present invention are directed to generating electrode stimulation signals for stimulating cochlear tissue using an implanted electrode array. An acoustic audio signal is processed to generate band pass signals which represent an associated band of audio frequencies. Stimulation information is extracted from the band pass signals to generate stimulation event signals that define electrode stimulation signals. The stimulation event signals are then weighted according independent channel-specific loudness functions to produce electrode stimulation signals. The electrode stimulation signals are developed into output electrode pulses to the electrodes in the implanted electrode array for stimulating cochlear tissue.

In specific embodiments, the loudness functions may be logarithmic functions. The loudness functions may reflect ISO normal hearing contours, or a loudness percept determined with respect to a basal-most electrode in the implanted electrode array, or psychoacoustic hearing factors, for example, from a post-implant patient fitting process.

### BRIEF DESCRIPTION OF THE DRAWINGS

FIG. 1 shows major signal processing blocks of a typical cochlear implant system.

FIG. 2 illustrates typical implementation of channel specific loudness mapping based on a single logarithmic function.

FIG. 3 illustrates the general functional form of channel specific loudness mapping as done in existing cochlear implant systems.

FIG. 4 shows various equal-loudness contours according to ISO 226:2003 for a normal hearing person.

FIG. 5 charts stimulation current at threshold, maximum loudness, and “half loudness.”

FIG. 6 charts stimulation current at threshold and “half loudness” in dB relative MCL.

FIG. 7 shows a typical implementation of independent channel specific loudness mapping according to an embodiment of the present invention based on use of a logarithmic function.

FIG. 8 shows the general functional form of independent channel specific loudness mapping according to an embodiment of the present invention.

### DETAILED DESCRIPTION OF SPECIFIC EMBODIMENTS

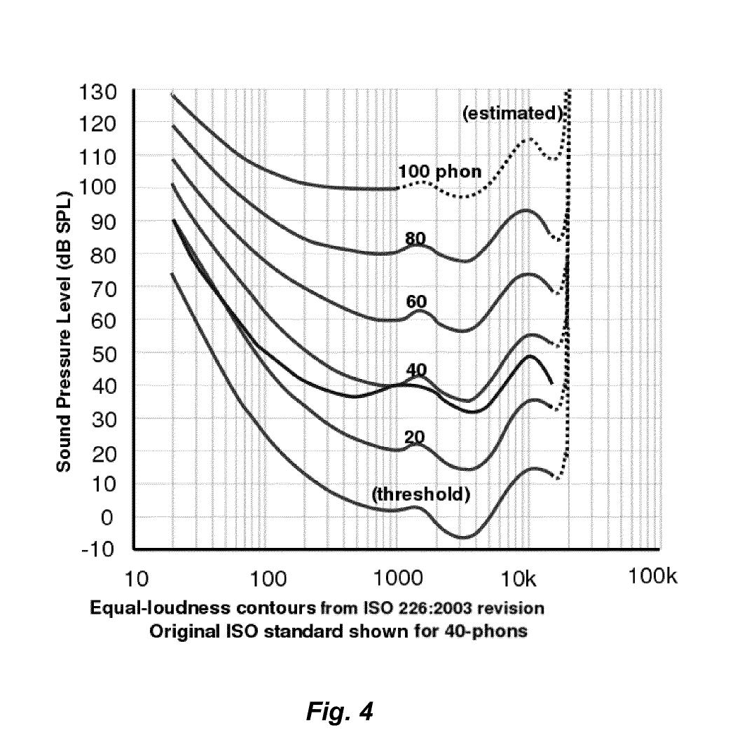

Existing CI systems do not take into account psychoacoustical properties of normal hearing such as the equal-loudness contours shown is FIG. 4. In normal hearing subjects, the acoustical level difference in loudness curves between the 20 phon and 100 phon is noticeably different between high frequencies and low frequencies. For normal hearing, loudness grows faster at 80 Hz than at 1000 Hz, so that where the difference in phons at 80 Hz amounts to just 57 dB SPL, at 1000 Hz the difference is about 80 dB SPL.

This effect has not been accounted for in existing CI systems, and little or no effort has been made to correctly code the acoustic intensity or sound level across frequencies and thereby stimulation sites. Comfortable loudness levels are typically defined based on a post-implantation fitting process that determines a hearing threshold (THR or T-level) for the user and a level at which maximum comfortable/acceptable loudness is reached (MCL or M-level). MCLs can vary from channel to channel for a CI user, and fitting MCLs does not necessarily lead to an equal loudness percept on all channels. Individual signal channels can be uncomfortable for various reasons and percepts, not just based on loudness.

During existing post-implant fitting procedures, loudness mapping functions are rarely changed even though beneficial effects on speech understanding and naturalness of perceived sounds could be expected. Hoth S., Indication For The Need Of Flexible And Frequency Specific Mapping Functions In Cochlear Implant Speech Processors. Eur. Arch. Otorhinolaryngol 264:129-138 (2007) (incorporated herein by reference) describes a subjective categorical loudness scaling procedure which could be used to determine an optimum frequency specific loudness mapping. Hoth concludes that the loudness mapping function of a CI can be optimized by individually scaling the loudness of electric and acoustic stimuli. The procedure he describes is rather time consuming and is limited to CI systems that do not allow channel specific loudness mapping.

U.S. Patent Publication 20070043403 describes a method of processing sound signals for an auditory prosthesis that uses a model of loudness perception by people with normal hearing. The current level of a first stimulation electrode is determined so that the loudness matches a normal hearing perception model for loudness. The electrode excitation pattern is then recalculated and the steps are repeated for each remaining electrode. Thus amplitude adjustments are individually performed on each electrode based on a loudness perception model using the concept of excitation patterns. This iterative procedure ends with a final excitation pattern that should provide a more natural loudness percept.

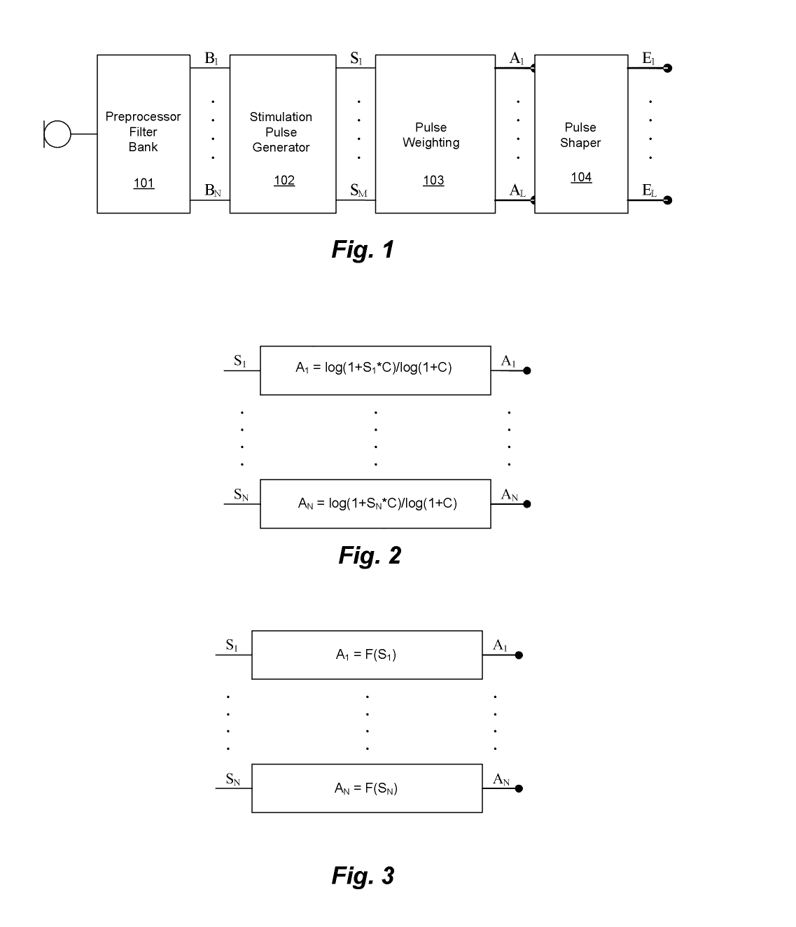

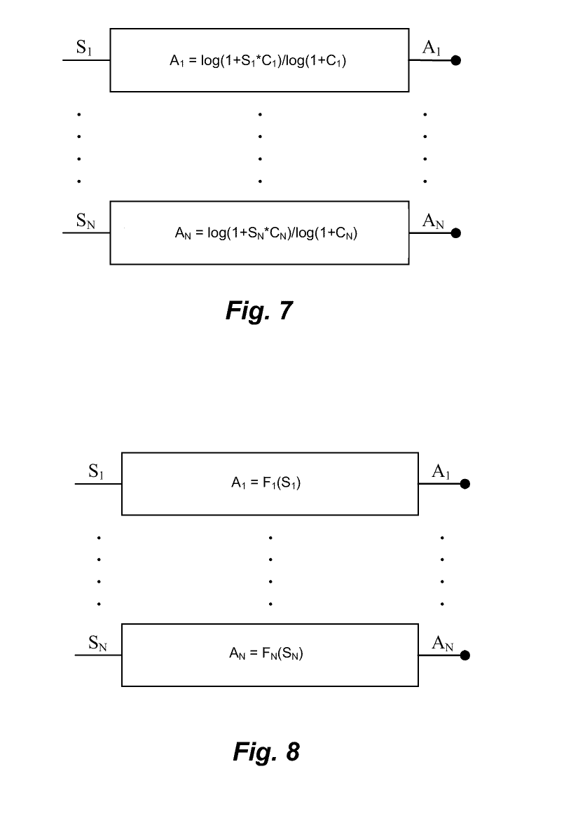

Embodiments of the present invention extend the signal processing arrangement for cochlear implants of the Pulse Weighting Module 103 as shown in FIG. 1 to provide channel specific amplitude mapping so that the stimulation event signals S₁ to S_{N} are weighted according independent channel-specific loudness functions to produce the electrode stimulation signals A₁ to A_{M}. For example, as shown in FIG. 7, the channel specific amplitude mapping can be defined by a logarithmic function with at least one set of parameters (C₁ to C_{N}) for the individual adjustment of frequency-specific and/or signal-specific loudness growth:

A_{N}=log(1+S_{N}*C_{N})/log(1+C_{N})

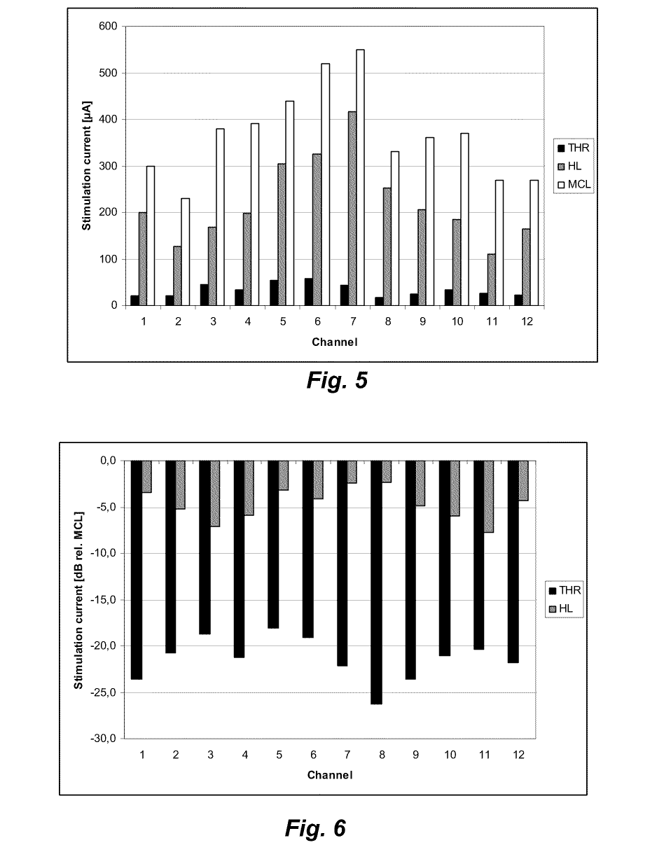

Parameters of specific loudness functions can be determined, for example, based on a categorical loudness scaling as already used in audiology testing. This results in at least one additional stimulation current contour such as the half-loudness contour HL shown in FIG. 5. The logarithmic functions may specifically reflect ISO normal hearing contours, or a loudness percept determined with respect to a basal-most electrode in the implanted electrode array, or psychoacoustic hearing factors, for example, from a post-implant patient fitting process.

An MCL contour that resembles the loudness percept of normal hearing subjects could be determined by presenting an acoustic stimuli at the most basal electrode at a level that results in stimulation at MCL of that channel, e.g. 90 dB SPL. The most basal electrode transmits the frequencies where normal hearing is least sensitive to sound pressure (see FIG. 5), so it is unlikely that an isophone with this level will exceed the comfortable level of the other electrodes. The neighboring electrode could be acoustically stimulated with a level that corresponds to the 90 dB SPL ISO loudness contour. The stimulation current could be adjusted until the subject perceives the stimuli with the same loudness as the previous electrode. This procedure would be repeated with every electrode, each time the stimulating current would be adjusted to the neighboring electrode. A second contour could be measured at subjective “half loudness” (HL) relative to the MCL contour.

FIG. 6 compares the THR and HL levels from FIG. 5 in terms of dB relative to MCL. Largely different ratios between THR and HL can be seen (e.g. channels 3 and 8). The values of C₁ to C_{N} could then be calculated from the three contours. Accordingly, alternative loudness mapping functions F₁ to F_{N} besides the specific logarithmic function shown in FIG. 7 could be used to parameterize loudness on different channels, as shown in FIG. 8.

Loudness perception of different acoustic sounds can be adjusted by individually adjusting mapping functions in different frequency bands. Thus loudness can be balanced across channels not only at hearing thresholds THR and maximum comfortable loudness MCL, but also at intermediate sound amplitudes, e.g. at “half loudness” HL. In addition, ISO loudness contours known from normal hearing may be modeled. Losses in speech understanding due to unbalanced stimulation amplitudes also may be resolvable. Fitting of the system can be performed relatively easily, for example, using categorical loudness scaling or loudness balancing at half loudness relative to one channel which is similar to balancing of MCLs. Besides relatively easy fitting arrangements, complex algorithms also are avoided such as those described in application US 2007/0043403 A1.

Embodiments of the invention may be implemented in any conventional computer programming language. For example, preferred embodiments may be implemented in a procedural programming language (e.g., “C”) or an object oriented programming language (e.g., “C++”, Python). Alternative embodiments of the invention may be implemented as pre-programmed hardware elements, other related components, or as a combination of hardware and software components.

Embodiments can be implemented as a computer program product for use with a computer system. Such implementation may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or analog communications lines) or a medium implemented with wireless techniques (e.g., microwave, infrared or other transmission techniques). The series of computer instructions embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product).

Although various exemplary embodiments of the invention have been disclosed, it should be apparent to those skilled in the art that various changes and modifications can be made which will achieve some of the advantages of the invention without departing from the true scope of the invention.

What is claimed is:

1. A method of generating electrode stimulation signals for an implanted electrode array, the method comprising:
processing an acoustic audio signal to generate a plurality of band pass signals each representing an associated band of audio frequencies;
extracting stimulation information from the band pass signals to generate a set of stimulation event signals defining electrode stimulation signals;
weighting the stimulation event signals according independent channel-specific loudness functions to produce a set of electrode stimulation signals; and
developing the electrode stimulation signals into a set of output electrode pulses to the electrodes in the implanted electrode array.

2. A method according to claim 1, wherein the loudness functions are logarithmic functions.

3. A method according to claim 1, wherein the loudness functions reflect ISO normal hearing contours.

4. A method according to claim 1, wherein the loudness functions reflect a loudness percept determined with respect to a basal-most electrode in the implanted electrode array.

5. A method according to claim 1, wherein the loudness functions reflect psychoacoustic hearing factors.

6. A method according to claim 1, wherein the loudness functions reflect a post-implant patient fitting process.

7. A computer program product implemented in a computer readable storage medium for generating electrode stimulation signals for a plurality of stimulation electrodes in an implanted electrode array, the product comprising:
program code for processing an acoustic audio signal to generate a plurality of band pass signals each representing an associated band of audio frequencies;
program code for extracting stimulation information from the band pass signals to generate a set of stimulation event signals defining electrode stimulation signals;
program code for weighting the stimulation event signals according independent channel-specific loudness functions to produce a set of electrode stimulation signals; and
program code for developing the electrode stimulation signals into a set of output electrode pulses to the electrodes in the implanted electrode array.

8. A product according to claim 7, wherein the loudness functions are logarithmic functions.

9. A product according to claim 7, wherein the loudness functions reflect ISO normal hearing contours.

10. A product according to claim 7, wherein the loudness functions reflect a loudness percept determined with respect to a basal-most electrode in the implanted electrode array.

11. A product according to claim 7, wherein the loudness functions reflect psychoacoustic hearing factors.

12. A product according to claim 7, wherein the loudness functions reflect a post-implant patient fitting process.

13. A system for generating electrode stimulation signals for an implanted electrode array, the system comprising:
means for processing an acoustic audio signal to generate a plurality of band pass signals each representing an associated band of audio frequencies;
means for extracting stimulation information from the band pass signals to generate a set of stimulation event signals defining electrode stimulation signals;
means for weighting the stimulation event signals according independent channel-specific loudness functions to produce a set of electrode stimulation signals; and
means for developing the electrode stimulation signals into a set of output electrode pulses to the electrodes in the implanted electrode array.

14. A product according to claim 13, wherein the loudness functions are logarithmic functions.

15. A system according to claim 13, wherein the loudness functions reflect ISO normal hearing contours.

16. A system according to claim 13, wherein the loudness functions reflect a loudness percept determined with respect to a basal-most electrode in the implanted electrode array.

17. A system according to claim 13, wherein the loudness functions reflect psychoacoustic hearing factors.

18. A system according to claim 13, wherein the loudness functions reflect a post-implant patient fitting process.
